(19) United States
(12) Patent Application Publication
(10) Pub. No.: US 2003/0082549 A1
(43) Pub. Date: May 1, 2003

(54) Method for determining alleles

(76) Inventor: Xiangjun Liu, Menomonee Falls, WI (US)

Correspondence Address: FOLEY & LARDNER, 150 EAST GILMAN STREET, P.O. BOX 1497, MADISON, WI, 53701-1497, US

(21) Appl. No.: 09/943,416

(22) Filed: Aug. 30, 2001

Related U.S. Application Data
(60) Provisional application No. 60/228,994, filed on Aug. 30, 2000.

(51) Int. Cl. C12Q 1/68; C07H 21/04

(52) U.S. Cl. 435/6; 536/25.4

(57) ABSTRACT

The present invention provides methods and kits for separating and identifying alleles, and thereby the haplotype, in genomic DNA samples. The method generally involves hybridizing primers specific to polymorphic sites within the alleles to the DNA sample, elongating the primers by one or more nucleic acids, separating the elongated primers and identifying the alleles utilizing the elongated primer. The method also allows for a ligation of two primers, their separation and subsequent use in identifying the targeted allele. The method further provides that another primer can be used as a blocking site for elongation of the first primer such that a stretch of DNA that includes a polymorphic site is replicated and identified. The unextended or extended primers can be labeled so that the primer can be easily separated and/or identified.

### CLAIM OF PRIORITY

This application claims priority from U.S. Provisional Patent Application No. 60/228,994, filed Aug. 30, 2000.

### TECHNICAL FIELD

The present invention relates to methods for separating and determining the identity of an allele by identifying one or more heterosequence sites in a gene. More particularly, the present invention relates to methods which utilize one or more primers for separating and determining the identity of an allele.

### BACKGROUND

The most frequent form of sequence variations among individuals are single nucleotide polymorphisms, popularly known as SNPs. With the completion of the Human Genome Project, SNPs are estimated to occur on an average of 1 out of every 1000 nucleotides but can occur more frequently in certain DNA regions. Efforts are now being focused on the use of SNPs to identify target genes associated with disease or drug response. However, due to weak correlations, many scientists and researchers challenge the idea of personalizing drugs and diseases based on an individual SNP, and so the importance of Haplotype analysis emerges as a critical tool to the medical utility of SNPs.

A haplotype is commonly known as the manner in which individual SNPs are organized along a given stretch of DNA. The classical definition of a haplotype is a combination of alleles of closely linked loci that are found in a single chromosome and tend to be inherited together from one generation to the next in a given population. Another aspect of molecular haplotyping is linkage disequilibrium mapping which is now recognized as an important tool in the positional cloning of disease genes, and numerous applications will become apparent as complex phenotypes are dissected genetically.

Since 1989 scientists have investigated various methodologies for molecular haplotyping using either single molecule dilution (SMD) of genomic DNA to separate alleles physically or allele discrimination by allele-specific primers to amplify selectively hemizygous DNA segments from a heterozygous template. However, these methods were developed for short segments only (approx. 500 bp), but more recently molecular haplotyping has been applied on long range PCR for markers 10-20 times farther apart and used the CD4 locus as a prototype system for the development of this assay. Other methods have been attempted to determine the haplotype of DNA sequences, however these methods have been largely unsuccessful, unreliable or expensive. Thus there remains a need for economic molecular haplotyping that is amenable to high throughput volumes that is reliable.

### SUMMARY OF THE INVENTION

The present invention is drawn to methodologies for determining alleles by identifying one or more heterosequence sites in a gene. The methodologies can be used to determine the haplotype of a specific gene, and has application in a number of areas, including human leukocyte antigen (HLA) typing. The present invention is also drawn to kits for such typing.

The present invention includes a method of separating allele specific nucleic acid molecules. One or more heterosequence site specific nucleic acid primers are added to single stranded nucleic acid molecules containing one or more heterosequence sites and allowed to hybridize. In one embodiment, the 3′ end of each primer corresponds to a polymorphic site of the targeted heterosequence site. In such embodiment, the 3′ end may be subjected to single base extension, ligation to a second primer having a 5′ end adjacent to the 3′ end of the heterosequence site specific primer or may be elongated for a number of bases. The elongated or ligated heterosequence site specific hybridized primer and nucleic acid molecules are then separated, and optionally recovered for further genotyping. In an alternative embodiment, each primer contains one or more polymorphic bases located within the primer such that primers which hybridize with less than 100% complementary bases can be selectively removed, and those primers which have hybridized with 100% complementary bases be unaffected.

The invention also relates to a method for identifying multiple alleles in a nucleic acid molecule containing such alleles. A single stranded nucleic acid molecule containing multiple heterosequence sites is selected. To this nucleic acid molecule two primers are added, a hetero primer and a homo primer. The hetero primer is capable of hybridizing to a 3′ heterosequence site that is located 3′ of a 5′ heterosequence site on the same nucleic acid molecule. The 3′ base of the hetero primer corresponds to a polymorphic base of the heterosequence site, such that elongation will only occur when the 3′ end of the hetero primer is hybridized to the single stranded nucleic acid. The homo primer is capable of hybridization to the same nucleic acid molecule at a position located 5′ of the 5′ heterosequence site. The primers are hybridized to the nucleic acid molecule, and the hetero primer is elongated such the 5′ hetereosequence site of the nucleic acid molecule located between the primers is replicated, that is the homo primer acts to stop elongation of the elongated hetero primer when it reaches the homo primer. The nucleic acid molecule and elongated hetero primer are denatured, and the hetero primer separated and analyzed to determine the 5′ heterosequence site. This information is used to identify a new set of nucleic acid primers containing another hetero primer and another homo primer, the hetero primer of the new set capable of hybridizing to the 5′ hetereosequence site (with the 3′ base of the hetero primer corresponding to a polymorphic base), the 5′ heterosequence site located 3′ to a further hetereosequence site on the same nucleic acid molecule, and the homo primer of the new set capable of hybridization to the same nucleic acid molecule at a position located 5′ of the further heterosequence site. The previous steps are repeated, with each new set of primers used in the subsequent round of hybridization/elongation until sufficient heterosequence sites on the nucleic acid molecule have been identified to identify the allele. The haplotype of the nucleic acid molecule may be determined in this manner.

The present invention also relates to a method for identifying multiple alleles in a nucleic acid molecule that comprises adding a nucleic acid sample containing multiple alleles to a set of beads, each bead having two distinct primers attached, at least one primer on each bead being a primer to a unique allele, under conditions such that at least the one primer to a unique allele hybridizes to a portion of the nucleic acid sample. The hybridized primer is amplified to extend the hybridized primer to produce an extended primer nucleic acid. The hybridized nucleic acid sample and primer are then denatured, and the nucleic acid sample removed from the beads. The extended primer is then hybridized to the second primer on the bead and the second primer is amplified. The beads containing the dual amplified primers are then analyzed to determine the alleles present in the nucleic acid sample.

### BRIEF DESCRIPTION OF THE DRAWINGS

FIG. 1 is a diagram which illustrates allele identification utilizing an allele specific primer extension methodology according to the present invention.

FIG. 2 is a diagram which illustrates a method of identifying multiple alleles using a single base extension with a primer size tag approach.

FIG. 2A is a diagram which illustrates a method of identifying multiple alleles using a single base extension with a primer size tag approach.

FIG. 3 is a diagram which illustrates allele identification utilizing allele specific ligation and primer size tags according to the present invention.

FIG. 4 is a diagram which illustrates allele identification utilizing hybridization and primer size tags according to the present invention.

FIG. 5 is a diagram which illustrates a method of identifying multiple alleles using sets of homo primers and hetero primers according to the present invention.

FIGS. 6A-6F illustrate a method of identifying multiple alleles using fluorescent beads comprising multiple primers according to the present invention.

### DETAILED DESCRIPTION OF THE INVENTION

The present invention is directed to a method for determining the identity of alleles, based on U.S. Provisional Patent Application No. 60/228,994, the entire content of which is hereby incorporated by reference.

The following terms are used throughout the application, and are defined as follows:

Allele: A variant form of a given gene. Such variants include single nucleotide polymorphisms, insertions, inversions, translocations and deletions.

Avidin: A family of proteins functionally defined by their ability to bind biotin with high affinity and specificity. Avidins are fairly small oligomeric proteins, made up of four identical subunits, each bearing a single binding site for biotin. Avidins can therefore bind up to four moles of biotin per mole of avidin. Avidins include proteins (a) produced by amphibians, reptiles and avians, which is present in their eggs and known as avidin, and (b) produced by a streptomyces, Streptomyces avidinii, and known as streptavidin. As used herein “avidin” includes all of the above proteins.

Biotin: As used herein, “biotin” includes biotin, commercial biotin products in which the biotin has been modified by the addition of alkyl groups, and biotin derivatives such as active esters, amines, hydrazides and thiol groups with the complimentary reactive groups on polymers being amines, acyl and alkyl leaving groups, carbonyl groups and alkyl halides or Michael-type acceptors.

Detection Molecule: A molecule covalently attached to a nucleic acid that allows for detection and/or removal of the nucleic acid, typically by an external source. Such molecules may comprise dyes, variable weight molecules including poly A and poly T tails, linkers which may be connected to beads including magnetic beads, biotin, avidin, digoxigenin, digoxigenin antibodies and other similar materials well known in the art.

Genotype: The particular alleles carried at a genetic locus.

Haplotype: Denotes the collective genotype of a number of closely linked loci and is the complete sequence of alleles along the same chromosome.

Hetero primer: A primer which will hybridize under stringent conditions to one unique allele.

Heterosequence site: Two alleles that have different sequences at a defined sequence site are said to have a heterosequence site.

Homo primer: A primer that will hybridize to both parental alleles.

Parental Alleles: Alleles from mammalian diploid cells which contain one set of chromosomes from the maternal side and one set of chromosomes from the paternal side.

Primer: An oligonucleotide which can be hybridized to a DNA template.

All patents and references cited herein are hereby incorporated by reference.

The methods of the present invention have several important advantages. The methods of the present invention allow for quick, inexpensive, accurate determination of alleles, including complete genotype and haplotype determinations. The methods will allow for analysis of nucleic acid fragments having lengths that prevent complete amplification by standard amplification means known in the art, such as the polymerase chain reaction

The present invention is directed to methods of separating and identifying allele specific nucleic acid molecules. Any nucleic acid molecules may be used, with deoxyribonucleic acids being preferred. The allele specific nucleic acid molecules that may be identified and separated include alleles of polyallelic genes, segments of genes and non-expressed fragments.

The methods and kits of the present invention may be used with all diploid genetic material which has two or more heterosequence sites, thus having multiple types of alleles. Examples of genes with multiple alleles to which the invention may be applied are the mammalian MHC genes such as human leukocyte antigen (HLA) class I and class II genes, the T cell receptor genes in mammals, TAP, LMP, ras, non-classical HLA class I genes, the genes for human complement factors C4 and C2, Bf in the human HLA complex, and genes located in mitochondrial DNA, bacterial chromosomes and viral DNA.

In one method of the present invention, a nucleic acid sample containing multiple alleles is obtained, each allele having a unique set of heterosequence sites. The nucleic acid sample is amplified by any means well known in the art, in one embodiment by the polymerase chain reaction (PCR), as described in Mullis, U.S. Pat. No. 4,683,202, issued, Jul. 28, 1988. The amplified nucleic acid sample is then denatured into single stranded nucleic acid. This single stranded nucleic acid may then be analyzed to determine the alleles present by determining the hetereosequence sites present by a number of approaches according to the present invention.

The methods according to the present invention utilize one or more primers. Primers according to the invention comprise a sequence of nucleotides that will hybridize with the sequence of interest. In some cases, it is required that the primers hybridize under conditions so that the primer will be capable of being elongated during amplification. In other cases, it is required that primers that are a 100% complementary match when hybridized have a higher Tₘ than primers that hybridize with less than a 100% complementary match. In general, the primers of the present invention can be any useful length, but will generally contain from about 12 to 25 nucleotides or at least 18 nucleotides, with a preferred length of about 18 to 22 nucleotides. In the methods of the present invention, it is necessary to identify one or more primer sequences unique for the target DNA within the sample so as to identify the polymorphic sites of interest. Such polymorphic identification of many multiple allele genes are known in the art. For example, there are about 222 known alleles of the HLA-A, HLA-B and HLA-C genes and the sequences of such alleles are well known in the art. See Arnett and Parham, Tissue Antigens 45: pp. 217-257, 1995, and Baxter-Lowe et al., U.S. Pat. No. 5,702,885, issued Dec. 30, 1997.

The expression “hybridize under highly stringent conditions” to describe the hybridization of nucleic acid molecules encompassed within the scope of this invention refers to hybridizing under conditions of low ionic strength and high temperature for washing. The expression “hybridize under low stringency” refers to hybridization conditions having high ionic strength and lower temperature.

Variables affecting stringency include, for example, temperature, salt concentration, probe/sample homology and wash conditions. Stringency is increased with a rise in hybridization temperature, all else being equal. Increased stringency provides reduced non-specific hybridization. i.e., less background noise. “High stringency conditions” and “moderate stringency conditions” for nucleic acid hybridizations are explained in Current Protocols in Molecular Biology, Ausubel et al., 1998, Green Publishing Associates and Wiley Interscience, NY, the teachings of which are hereby incorporated by reference. Of course, the artisan will appreciate that the stringency of the hybridization conditions can be varied as desired, in order to include or exclude varying degrees of complementation between probe and analyte, in order to achieve the required scope of detection.

Various detection molecules may be used in the present invention. These molecules may be coupled to one or more primers, or may be coupled directly to ddNTPs that are incorporated into nucleic acids during elongation steps. These molecules may comprise a means for detecting the molecule, such as dyes, radiolabels, etc., or they may comprise a means for separating the molecules, such as biotin/avidin, magnetic and/or fluorescent beads, etc., or both. For example when biotin/avidin are used, one or more of the primers may be labeled with biotin, so that when the primers are hybridized to single stranded nucleic acids, the resultant double stranded DNA is produced in which one strand carries a biotin label. The double stranded DNA may then be bound to a solid support coated with avidin.

The solid support used in the invention may be any such support well known in the art such as a bead, an affinity chromatography column. A preferred support is in the form of a magnetic bead. When the support is in the form of a bead, the two strands of the amplified nucleic acid are separated by attracting the beads to a magnet and washing the beads under conditions such that the double stranded nucleic acid dissociates into single strands of nucleic acid. The dissociation is typically performed by incubating the beads in several repetitions under alkaline conditions, typically 0.1 M or 0.15 M NaOH, at room temperature for about 5 to 10 minutes. Either strand can then be collected and further analyzed.

Various analysis techniques can be used to identify the isolated heterosequence sites to determine the alleles. These techniques are well known in the art and include, but are not limited to, electrophoresis such as polyacrylamide gel electrophoresis, flow cytometery, high pressure liquid chromatography laser scanning and mass spectroscopy. These techniques can be done manually or by an automated system. Such automated systems are well known in the art and include an automated sequencing machine or capillary electrophoresis machine which are able to scan multiple-color fluorescence.

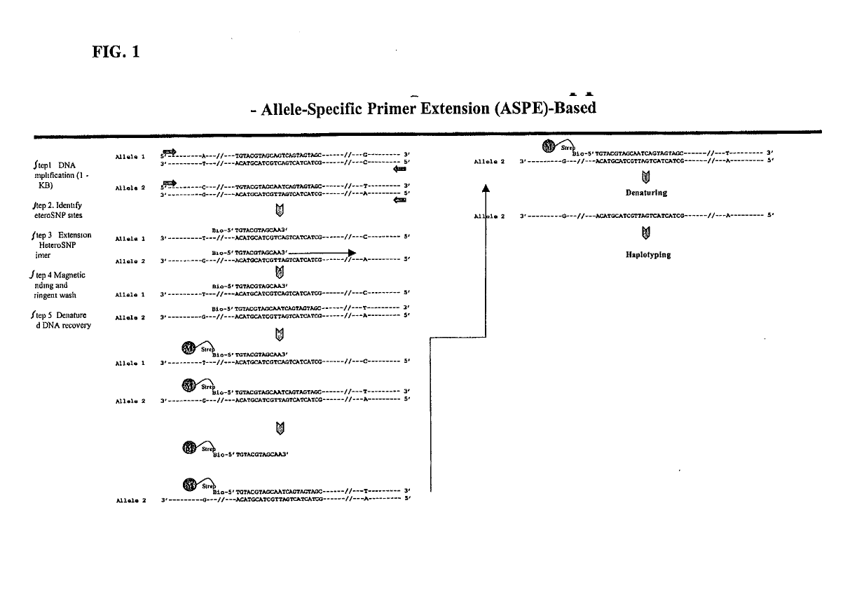

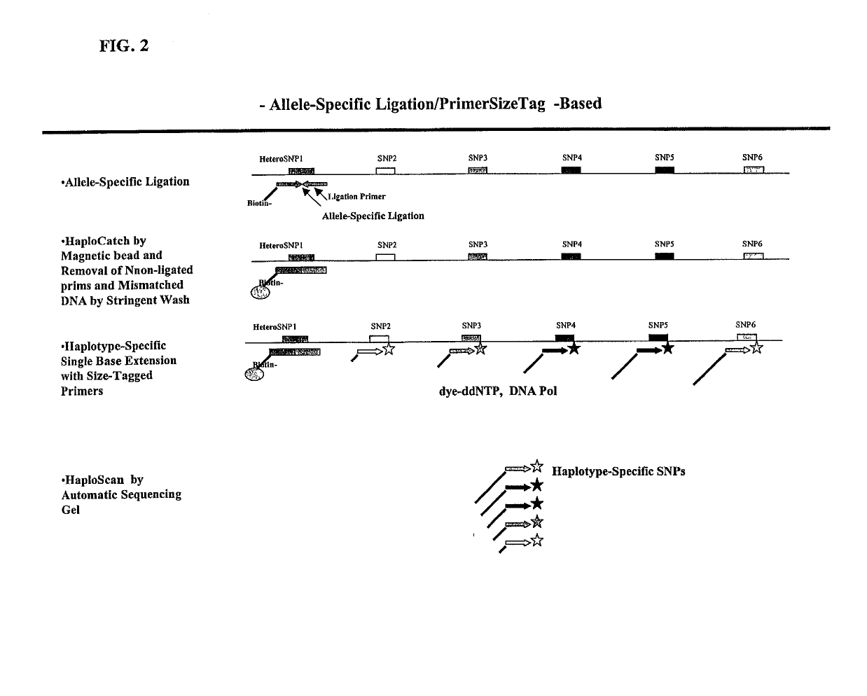

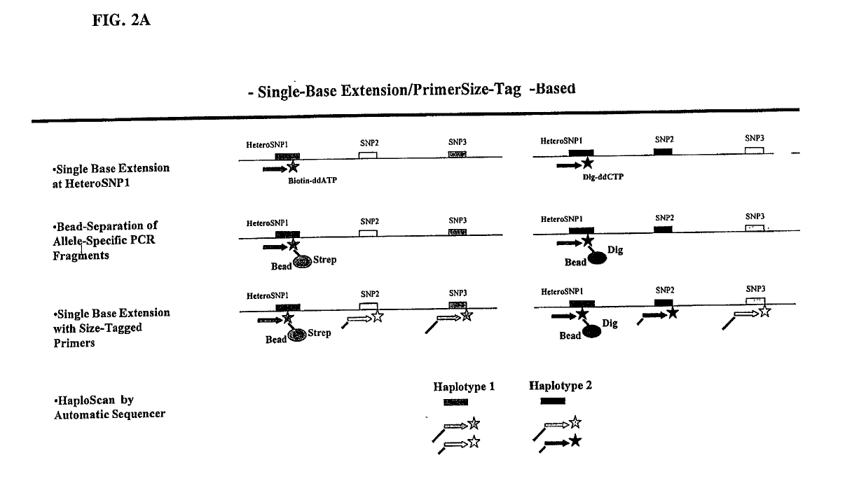

The first approach of the present is diagrammed in FIGS. 1 and 2 and relies on elongation of hybridized heterosequence site specific primers. This approach is particularly useful to determine allele or haplotype-specific genotype information in a highly polymorphic chromosome region. As shown in FIG. 1, following amplification and denaturing of a DNA sample to produce single stranded DNA fragments, one or more heterosequence site specific primer(s) which is labeled with a detection molecule at the 5′ end is added. The heterosequence site specific primer is added to the single stranded nucleic acid molecule and allowed to hybridize. In a preferred embodiment, the 3′ end of each primer is complementary to a polymorphic base of a heterosequence site. Therefore, if the primer hybridizes to a heterosequence site wherein the 3′ base is not complementary, the primer will not undergo elongation when subjected to conditions for elongation. Preferably an enzyme that is capable of distinguishing single nucleotide differences is utilized. As shown in FIG. 1, the hybridized primers are then subjected to elongation, with only the primers which have hybridized with complementary 3′ base matches being elongated. The primers are then removed via the detection molecule, exemplified as biotin in FIG. 1. Magnetic beads coated with avidin are used to remove the primers via the biotin on the primers. The hybridized primer/DNA fragments are then washed under conditions such that the DNA fragments bound to those primers that have not undergone elongation are removed. The elongated double stranded nucleic acids are then denatured. The strands not bound to the bead may then be analyzed to determine the heterosequence site(s).

Alternatively, the primers used in the invention may not be coupled to a detection molecule at their 5′ ends. Rather, the primers will be allowed to hybridized as previously described, and those that hybridize with complementary 3′ ends will be subjected to single base extension using ddNTPs that are coupled to detection molecules as shown in FIG. 2. The detection molecules on the extended primers will be used to separate the primers, and the primers can then be denatured and analyzed to determine the heterosequence site(s) present.

The present invention is also useful for high-throughput single nucleotide polymorphism typing using an automated sequencing machine or capillary electrophoresis machine which are able to scan four-color fluorescence when using the following method. The same method can also modified to typing other genetic variations other than single nucleotide polymorphisms, including multibase polymorphisms, insertions, inversions, translocations and deletions.

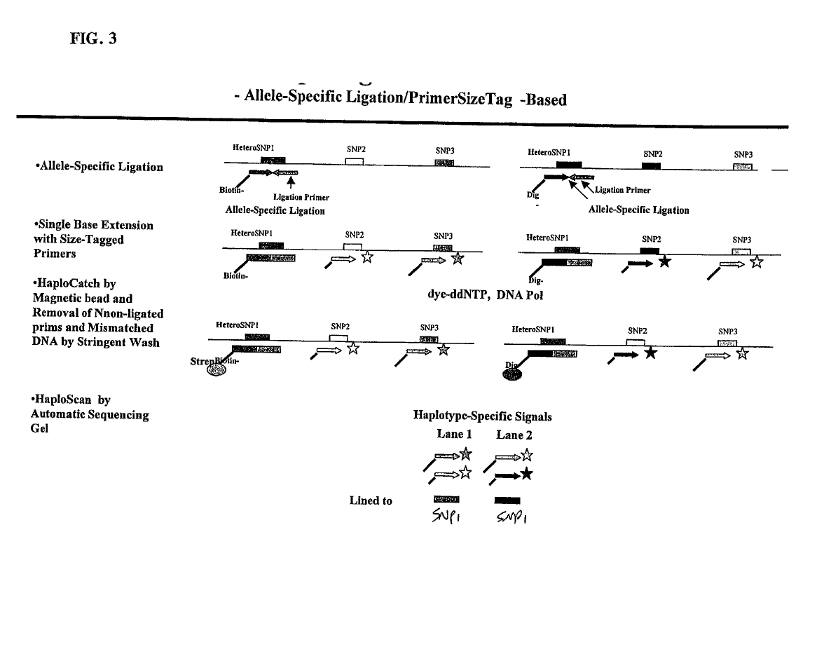

Another approach of the present invention relies on allele specific ligation. This approach is illustrated in FIG. 3. As shown in FIG. 3, heterosequence site specific primers are added to single stranded DNA fragments containing one or more heterosequence sites. The heterosequence specific primers have the 3′ end of each primer complementary to a polymorphic base of a heterosequence site and are allowed to hybridize to the DNA fragments. Ligation primers are then added, and allowed to hybridize to the DNA fragments. Each ligation primer has a sequence that is complementary to a portion of one of the DNA fragments, such that the 5′ end of the ligation primer is directly adjacent to the 3′ end of the heterosequence site specific primer. If the heterosequence site specific primer does not hybridize to the DNA fragment, the ligation primer will be unable to ligate to the heterosequence site specific primer when subjected to conditions for ligation. The primers are ligated, if possible, and then subjected to temperature conditions sufficient to denature the primers that have not ligated, but insufficient to denature ligated primers that have hybridized to the DNA fragments. Typically, such temperature will be approximately 60° C. when 20 mer primers are used. The ligated primers that have hybridized may then be removed by any means known in the art. As shown in FIG. 3, one set of the primers may have a detection molecule attached, illustrated as biotin. The detection molecule may be attached to the heterosequence specific primers or the ligation primers. Moreover, the methodologies as described may be combined, as shown in FIG. 3, and polymorphism at one heterosequence site detected by one method, and the other sites determined by other methodologies described herein. Also as shown in FIG. 3, one or more of the primers may have a variable weight molecule coupled to the 5′ end of each primer, such that no two primers have the same molecular weight. Such variable weight molecules can be any appropriate materials that are unreactive in the hybridization/amplification steps, and include poly homonucleic acid tails, such as poly A tails. Such poly A tails generally differ in length from 2 to 4 bases, but may be of any different length that is sufficient to separate such primers with poly A tails on standard separating equipment, such as gel electrophoresis.

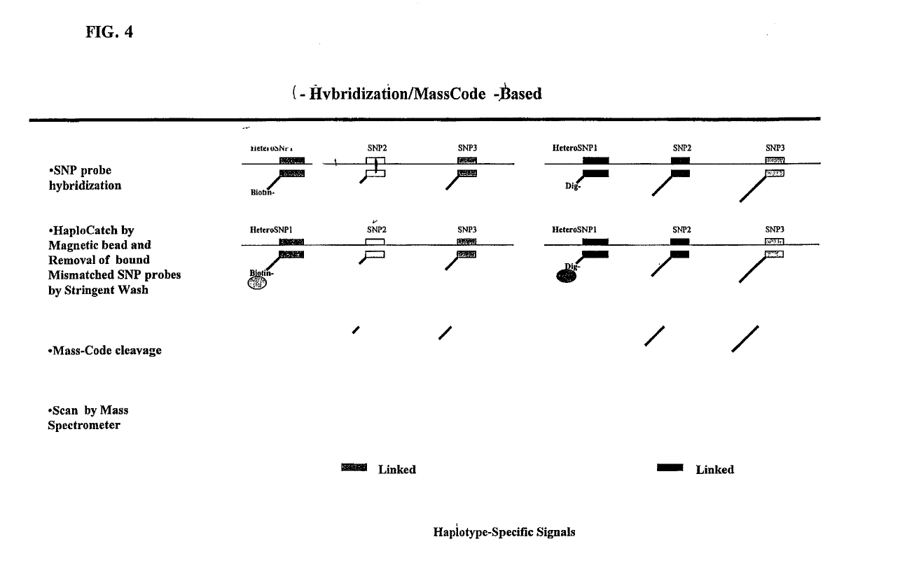

Another method of the present invention is illustrated in FIG. 4. According to such methodology, a set of heterosequence specific primers are added to DNA fragments containing multiple heterosequence sites. Each primer has at least one polymorphic base, located within each primer such that following hybridization of the primers to the DNA fragments, those primers that hybridize with base mismatches will have a lower Tₘ than those primers that hybridize without base mismatches. This difference in Tₘ is then used to a to remove those primers which have less than 100% complementary hybridization. Such base mismatches typically occur near the center of the primer sequence. After removal of the less than 100% complementary hybridization primer/DNA fragment conjugates, the remaining conjugates are analyzed to determine the specific heterosequence sites to determine the specific allele. This may be done in a variety of ways. As illustrated in FIG. 4, all primers may have a variable weight molecule attached. All primers for each specific heterosequence site may have a specific variable weight molecule attached. Each primer for each individual polymorphism at one or more specific heterosequence site will have a different detection molecule attached. By separating the hybridized primers into individual groups by the detection molecules, and by further determining which variable weight molecules are present in each group of primers, the allele specificity is determined.

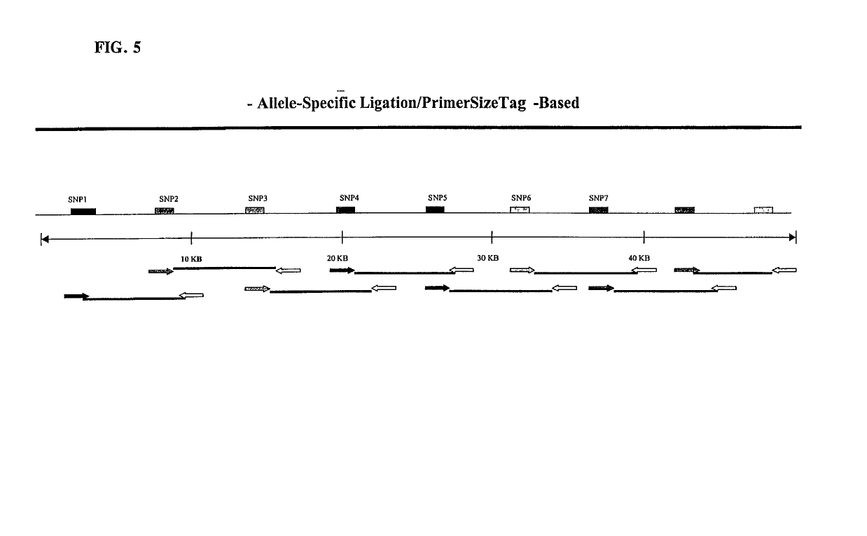

Another method of the present invention allows for the determination of multiple heterosequence sites on long segments of nucleic acid that may be too long to be fully amplified by traditional means such as PCR. As shown in FIG. 5, a single stranded nucleic acid molecule containing multiple heterosequence sites is selected. To this nucleic acid molecule two primers are added, a hetero primer and a homo primer. The hetero primer is capable of hybridizing to a 3′ heterosequence site that is located 3′ of a 5′ heterosequence site on the same nucleic acid molecule. The 3′ base of the hetero primer corresponds to a polymorphic base of the heterosequence site, such that elongation will only occur when the 3′ end of the hetero primer is hybridized to the single stranded nucleic acid. The homo primer is capable of hybridization to the same nucleic acid molecule at a position located 5′ of the 5′ heterosequence site. The primers are hybridized to the nucleic acid molecule, and hetero primer is elongated such that the 5′ hetereosequence site of the nucleic acid molecule located between the primers is replicated. The nucleic acid molecule and elongated hetero primer are denatured, and the hetero primer separated and analyzed to determine the 5′ heterosequence site. This information is used to identify a new set of nucleic acid primers containing a hetero primer and a homo primer, the hetero primer of the new set capable of hybridizing to the 5′ hetereosequence site (with the 3′ base of the hetero primer corresponding to a polymorphic base), the 5′ heterosequence site located 3′ to a further hetereosequence site on the same nucleic acid molecule, and the homo primer of the new set capable of hybridization to the same nucleic acid molecule at a position located 5′ of the further heterosequence site. The previous steps are repeated, with each new set of primers used in for the subsequent round of hybridization/elongation until sufficient heterosequence sites on the nucleic acid molecule have been identified to identify the allele. The haplotype of the nucleic acid molecule may be determined in this manner.

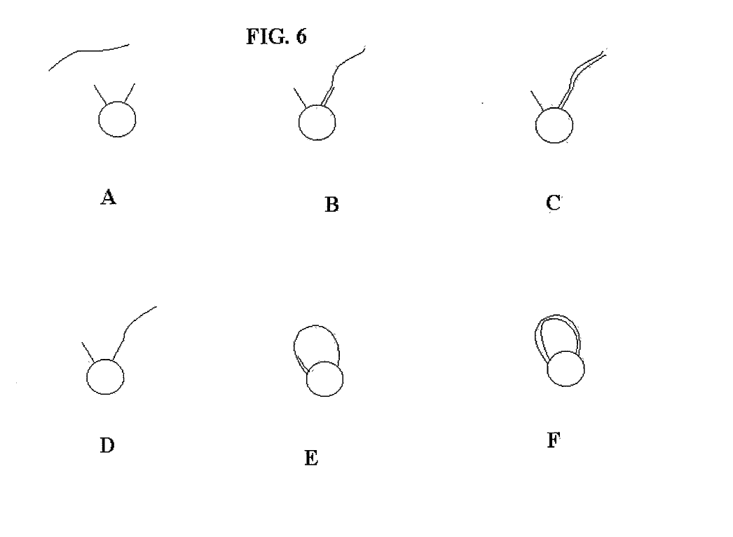

As shown in FIGS. 6A-6F, the present invention also relates to a method for identifying multiple alleles in a nucleic acid molecule. As shown in 6A, the method comprises adding a nucleic acid sample containing multiple alleles to a set of beads, each bead having two distinct primers attached, at least one primer on each bead being a primer to a unique allele. The nucleic acid is then reacted under conditions such that the at least one primer to a unique allele hybridizes to a portion of the nucleic acid sample as shown in 6B. The hybridized primer is amplified to extend the hybridized primer to produce an extended primer nucleic acid as in 6C. Moving to 6D, the hybridized nucleic acid sample and primer are then denatured, and the nucleic acid sample removed from the beads. The extended primer is then hybridized to the second primer on the bead (6E) and the second primer is amplified (6F). The beads containing the dual amplified primers are then analyzed to determine the alleles present in the nucleic acid sample. For easy removal of the primers from the beads the primers can have a cleavage site.

The present invention also embodies kits for carrying out the methods described herein. In their most basic embodiment the kits of the present invention comprise instructions for carrying out the methods discussed above. Additionally, the kits can contain at least one or more of the required reagents utilized in the present methods, such as one or more sets of locus specific amplification primers, polymerase chain reaction buffer, dideoxynucleotides, wherein one or more is optionally labeled, reagents for nucleic acid amplification, reagents for generation of single stranded nucleic acid fragments, one or more heterosequence site specific primers, optionally conjugated to at least one detection molecule, one or more ligation primers, reagents for ligation of adjacent hybridized primers, beads containing one or more detection molecules, and one or more sterile microtubes.

This invention will be better understood from the Examples which follow. However, one skilled in the art will readily appreciate that the specific methods and results discussed are merely illustrative of the invention and no limitation of the invention is implied.

### EXAMPLES

The present examples involved the use of three strategies to verify the capture of different alleles pertaining to a specific polymorphism in the HLA Gene: i) Hybridization; ii) Single Base Extension; and iii) Ligation

Each of these conditions were used as a test to develop an assay that would be helpful in identifying the appropriate allele and hence the specific polymorphism pertaining to that allele. The last two methods were enzyme based assays and required the use of a Taq Ligase, and a Thermus Sequenase that exploits the ability of these enzymes to distinguish single nucleotide differences at specific positions on a single stranded DNA. These methods have been noted to be sensitive enough to distinguish single nucleotide polymorphisms or mutations within specific alleles under investigation.

1.A. Hybridization

One method of detection was hybridization of a specific captured target to oligo coupled microspheres and assaying the complex. The reactions were set up as described below. Any allele to be captured was subject to 2 rounds of Hybridization. The first round of Hybridization used different homo and heterozygous DNA and specific oligo coupled bead that recognized a particular sequence. The second round of Hybridization used another set of beads that recognized a specific sequence within that target which confirmed the presence of the captured allele. However, a single round of hybridization was initially done as a control experiment to test the specificity of the oligo coupled microspheres to different alleles within a target.

A 158 bp DNA fragment of HLA-A locus was amplified using sense primer 5′ A200A and antisense primer 3′A322-1 with various genomic DNA samples obtained from UCLA registries (UCLA 210, UCLA 230 and UCLA 243). The 158 bp fragment was produced for this example using standard amplification methods. Primers used to amplify both Homo and Heterozygous DNAs in this example were:

| | |
|---|---|
| 5′A200A | |
| 5′-ACA GCG ACG CCG CGA GCC A-3′ | position 182-200, |
| | sense primer |
| 3′A322-1 | |
| 5′-CCTCGCTCTGGTTGTAGTA-3′ | position 322-340, |
| | antisense primer |

Single stranded DNA (ss) for use in ligation, single base extension or hybridization was generated by Asymmetric PCR. The conditions for the asymmetric PCR were as above, except the sense primer was added at 50 times lower concentration than the antisense primer. The antisense primer was biotinylated to generate a 5′ biotin-labeled single stranded PCR fragment.

Alternatively, the use of a 5′-3′ exonuclease, T7 gene 6 exonuclease, could be used to produce ssDNA. In this case, the strand of interest is protected through the introduction of 4 phoshothioate bonds at the 5′ end of the PCR primer during oligonucleotide synthesis. T7 exonuclease degrades the strand that does not contain the phoshothioate bases at the 5′ end of the primer.

1.B. Single Base Extension Reaction (SBER)

The Single Base Extension Reaction (SBER) of the present example utilized an extension primer which was designed so that the 3′ end annealed adjacent to the polymorphic base. The extension protocol of this example used either Thermosequenase, or the Klenow large Fragment polymerase to incorporate the polymorphic base in a cycling or a non-cycling reaction, respectively.

Using the single base extension reaction in an attempt to capture a specific allele; Allele Specific PCR was performed using Primer Mixes (PM), H001 and H002. These two primer mixes were used for the incorporation of specific bases at the site of the polymorphism. Both PM used a common 5′ primer(agcgacgccgcgagcca), but used an allele specific 3′ primer. PM H001 specifically incorporated the “C” (ccaagagcgcaggtcctcy) base whereas PM H002 was specific for “A” (ccaagagcgcaggtcctct) at the respective sites of polymorphism, when a heterozygous DNA was used.

The extension reaction was done as described above. The product from the extension reaction was purified and bound to streptavidin magnetic beads. The high binding affinity of streptavidin for biotin allowed for the rapid and efficient isolation of biotin-labeled target molecules. The complex was washed a number of times to eliminate the possibility of any unbound label that could be a factor which might influence the next step of experimentation.

A number of different samples were tested for verification of the captured allele by ASPCR. ASPCR using PMH001 and H002 were done in sets of 5. Experimental and negative controls of a typical extension reaction protocol were as follows: The experimental sample used either biotinylated A or C in the extension reaction. It was assumed that Sequenase would correctly incorporate the specific base and hence a correct signal from the specific allele caught, would be detected based on the primer mix used. The two negative controls had the same components in the SBER as the experimental samples except the ddNTPs A or C was eliminated from the reaction. Another negative control used only unlabeled ddNTPs A, C, G and T. The supernatants of the following sets of reactions were verified by ASPCR using primer mixes H001, and H002. The supernatants tested were in sets of 5 and were as follows: After extension, after binding of the extension product to the magnetic beads, after a number of washes, and after the product was eluted from the magnetic beads at high temperature.

### Cycling Reaction

Each 20 μl reaction used 100 ng of a single stranded (ss) DNA of the HLA A locus which was obtained after PCR amplification of Genomic DNA as described above; 2 μM of an extension primer, 125 nM each of the unlabeled dideoxy terminators (ddG, T, A or C), and 500 nM of a biotin-labeled ddNTP (either A or C), depending on the specific base to be incorporated at the site of the polymorphism, 10× Enzyme reaction buffer (diluted to 1× final concentration) and 5 units of the Sequenase enzyme were added to the reaction mixture. The reaction was cycled at 94° C. for 1 min, followed by 40 cycles of 94° C. for 10 sec; and 60° C. for 30 sec. A final extension cycle at 72° C. for 10 min with a hold at 4° C. was used as the extension profile in this example.

### Non-Cycling Reaction

When the Klenow Large fragment polymerase reaction was used for extension, the first step required hybridization of the extension primer to the single stranded DNA. 100 ng of ssDNA was annealed to 20 μM of an extension primer. The primer and DNA were mixed together at 90° C. for 5 min and then cooled to room temperature slowly, so that a hybrid formed. This process took about 1 hour. The next step involved the addition of specific unlabeled and labeled biotin ddNTPs (1.5 μM), with 5U of the Klenow Large Fragment, and incubated at 37° C. for 30 min. 1.5 μl of 0.5 M EDTA was added to the reaction mixture at the end of extension.

The extension product (cycling or non-cycling), was purified using a QIAQUICK® column (Qiagen), to remove the unincorporated biotin. 10 μl of Streptavidin coated Magnetic beads (in a 2× binding buffer 10 mM Tris pH 7.5, 1 mM EDTA, 2.0 mM NaCl) was mixed for 20 min at room temp with 20 μl of the purified extension product. A magnetic field was applied to the beads and the unbound extension product was discarded. The beads were washed at least twice with 1 ml of the same binding buffer, and the strand of interest was eluted from the beads by applying heat at 95° C. for 2 min.

The eluted strand was then subjected to Allelic specific PCR (ASPCR) using specific primers to confirm the polymorphism of that specific allele. Appropriate controls were implemented to confirm the result.

1.C. Ligation Method

This example involved the use of a ligation event between two primers before annealing to a single stranded DNA template. This example was performed with the understanding that ligation of the two primers with the ssDNA when perfectly matched would form a strong duplex and thus sustain a higher temperature washing (greater than the Tₘ of the primers). The mismatched template would find it difficult to withstand washing at temperatures higher than the Tₘ of the primers and would free itself form the duplex and ultimately wash off.

Two primers were placed adjacent to each other in which one primer, an allele specific or heterosequence primer, had a polymorphic site at the 3′ end and a biotin label at the 5′ end. The second primer was a ligation primer that had a phosphate group on the 5′ end to mediate ligation. It was assumed that both primers would ligate together before hybridizing to the ssDNA template although the present method does not depend on this assumption. The 20 μl reaction mixture contained 10 μl (100 ng) of a specific ssDNA, 1 μl of each of the primers (1 μM), 2 μl of a 10× Ligation Buffer and 10U of Taq Ligase.

The mixture was heated in a thermocyler at 90° C. for 2 min, followed by a 30 min incubation at 37° C. at which time the reaction was stopped by the addition of EDTA. The mixture was purified using a QIAQUICK® column to eliminate all unincorporated primer and biotin that can account for the non-specificity in an allele specific PCR reaction.

The purified complex was bound to streptavidin coated magnetic beads as described above. The complex was washed under high stringency washing conditions. Stringency of the wash was controlled by elevated temperatures of the wash buffer (55-95° C.), so a threshold temperature was be reached for the separation of the allele-specific DNA fragment. The eluted template was further verified by Allele specific PCR using primers that recognized the site of polymorphism of the captured allele.

2. Hybridization Assay for Haplotyping

Different oligonucleotides for specific polymorphisms of the HLA A Locus were coupled to different bead sets (Luminex) to be used in the hybridization assay. The template that hybridized to the oligo coupled beads was selected to provide perfect sequence homology. Coupling beads to specific oligos was performed according to the manufacturer's instructions (Luminex Corp.). The Luminex bead-probe conjugate were hybridized with PCR fragments produced above. The sequence of the probes used for separation of allele specific PCR fragments was:

| | |
|---|---|
| L5′A107A | 1AGGTATTTCTACACCTCCGTG |
| L5′A107C | 1AGGTATTTCTCCACATCCGTG |

The non-hybridized PCR templates were washed away and the PCR fragment specific hybridized to 5′A107A or 5′A107C were eluted from the Luminex beads. Oligos of different sizes, with and without a spacer (i.e. which contained an additional 20 random bases in the middle of an oligo sequence), were coupled to various bead sets and hybridized to different templates to assay for specificity of different alleles. The numbers in the primer identification correlate to different oligonucleotides coupled to beads and indicate the site of the polymorphism for a specific allele. For example, 107 A or C signifies the site of polymorphism at base 107 where each allele either has an A or a C at position 107.

The reaction protocol for hybridization was as follows: 17 μl of ssDNA was denatured at 95° C. for 5 min, followed by the addition of 33 μl of a specific oligo coupled bead (5000 beads/oligo), complementary to the template and incubated at 55 C. for 30 min. When the oligo with the spacer was used the hybridization temperature was increased to 65° C. to ensure specificity. The bead mixture was thoroughly vortexed and sonicated and brought up to the required hybridization temperature, before addition of the ssDNA. Following hybridization the mixture was centrifuged at 2000×g; washed twice with 1 ml each of 1.5× TMAC (3M TMAC, 0.1% SDS, 50 mM Tris-Cl, pH 8.0, 4 mM EDTA pH 8.0) and the supernatant was discarded.

20 μl of H₂O was added to the complex and the captured template which was bound to the oligo coupled bead was eluted at 95° C. for 5 min. 1 μl of the eluted template was subjected to asymmetric PCR to obtain a greater abundance of the eluted template for a second round of hybridization.

A second round of Hybridization was performed with a second bead set that was complementary to the captured template as a test to confirm the accuracy of the template. The samples were measured on a Luminex 100 flow cytometry instrument after the addition of 120 ng of Streptavidin-Phycoerythrin (SA-PE) to each tube and incubated at the hybridization temperature for another 5 minutes. The amount of fluorescent signal obtained was a true representation of the interaction of the biotin with the SA-PE. This assay was a quantitative one and the amount of positive signal was expressed as the highest number obtained for a given reaction.

The second round of hybridization used other allele-specific Luminex bead-probes as follows:

Luminex bead-probes used to confirm allele specific separation

| | |
|---|---|
| L5′A107A | 1AGGTATTTCTACACCTCCGTG |
| L5′A107C | 1AGGTATTTCTCCACATCCGTG |
| L5′A153A | 1CTTCATCGCAGTGGGCTAC |
| L5′A153C | 1CTTCATCGCCGTGGGCTAC |
| L5′A249T | 1GCAGGAGGGTCCGGAGTAT |
| L5′A249G | 1GCAGGAGGGGCCGGAGTAT |
| L5′A291C | 1GAAGGCCCACTCACAGACT |
| L5′A291G | 1GAAGGCCCAGTCACAGACT |

TABLE 1
Expected allele-specific reaction pattern after hybridization

| Template | Luminex bead-probe reaction pattern | | | | | | |
|---|---|---|---|---|---|---|---|
| DNA Name | HLA-A Allele | L5’A107A | L5’A107C | L5’A249G | L5’A249T | L5’A291C | L5’A291G |
| UCLA 210 | A*0206,− | + | − | − | + | + | − |
| (homozygote) | | | | | | | |
| UCLA 230 | A*2402101 | − | + | + | − | + | − |
| (heterozygote) | A*3401 | + | − | + | − | − | + |
| UCLA 243 | A*2402101,− | − | + | + | − | + | − |
| (homozygotes) | | | | | | | |

TABLE 2
Observed allele-specific reaction pattern hybridization.

| Template | | | | | | | |
|---|---|---|---|---|---|---|---|
| DNA Name | Probe | L5’A107A | L5’A107C | L5’A249G | L5’A249T | L5’A291C | L5’A291G |
| UCLA 210 | L5’A107A | (+) 166 | (−) 50 | (−) 124 | (+) 279 | (+) 234 | (−) 21 |
| (homozygote) | L5’A107C | (−) 152 | (−) 60 | (−) 137 | (−) 330 | (−) 223 | (−) 29 |
| UCLA 230 | L5’A107A | (+) 63 | (+) 111 | (+) 90 | (−) 56 | (+) 94 | (−) 27 |
| (heterozygote) | L5’A107C | (−) 52 | (+) 87 | (+) 70 | (−) 55 | (+) 57 | (−) 13 |
| UCLA 243 | L5’A107A | (−) 13 | (−) 57 | (−) 37 | (−) 23 | (+) 96 | (−) 14 |
| (homozygotes) | L5’A107C | (−) 15 | (+) 83 | (+) 60 | (−) 36 | (+) 124 | (−) 13 |
| Negative | | 7 | 9 | 14 | 19 | 6 | 9 |
| Control | | | | | | | |

TABLE 3
Observed allele-specific reaction pattern after hybridization using
negative control.

| Template | No Probe | | | | | | |
|---|---|---|---|---|---|---|---|
| DNA Name | (Control) | L5’A107A | L5’A107C | L5’A249G | L5’A249T | L5’A291C | L5’A291G |
| UCLA 210 | | (+) 65 | (−) 29 | (−) 65 | (+) 124 | (+) 97 | (−) 19 |
| (homozygote) | | | | | | | |
| UCLA 230 | | (+) 63 | (+) 111 | (+) 90 | (−) 56 | (+) 30 | (−) 68 |
| (heterozygote) | | | | | | | |
| UCLA 243 | | (−) 12 | (−) 216 | (−) 100 | (−) 23 | (+) 213 | (−) 10 |
| (homozygotes) | | | | | | | |
| Negative | | 7 | 9 | 14 | 19 | 6 | 9 |
| Control | | | | | | | |

The results in the tables above demonstrate successful allele-specific hybridization as the allele-specific numbers are higher than the non-allelic specific reactions.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like refer to ranges which can be subsequently broken down into subranges as discussed above.

While only a few, preferred embodiments of the invention have been described, those of ordinary skill in the art will recognize that the embodiment may be modified and altered without departing from the central spirit and scope of the invention. Thus, the preferred embodiments described above are to be considered in all respects as illustrative and not restrictive, the scope of the invention being indicated by the following claims, rather than by the foregoing description, and all changes which come within the meaning and range of equivalents of the claims are intended to be embraced.

The following references are hereby incorporated into the patent application in their entirety:

Jorde, L. B.: Am. J. Hum. Genet. 56, pp. 11-14, 1995;

Thomson, G.: Am. J. Hum. Genet. 57, pp. 474-486, 1995;

Ruano, G., Kidd, K. K. and Stephens, J. C.: Proc. Natl. Acad. Sci. USA 87, pp. 6296-6300, 1990;

Ruano, G. and Kidd, K. K.: Nucleic Acids Res. 19, pp. 6877-6882, 1991;

Beloin, S. M., Tishkoff, S. A., Bentley, K. L., Kidd, K. K. and Ruano, G.: Nucleic Acids Res. 24, pp. 4841-4843, 1996;

Gilles, P. N., Wu, D. J., Foster, C. B., Dillon, P. J. and Chanock, S. J.: Single nucleotide polymorphic discrimination by an electronic dot blot assay on semiconductor microchips. Nature Biotechnol. 17, pp. 365-370, 1999;

Little, D. P., Braun, A., O'Donnell, M. J. and Koster, H.: Mass spectrometry from miniaturized arrays for full comparative DNA analysis. Nature Med. 3, pp. 357-362, 1997;

Marshal, R. D., Koonts, J. and Sklar, J.: Detection of mutations by cleavage of DNA heteroduplexes with bacteriophage resolvases. Nature Genet. 9, pp. 177-183, 1995;

Nauck, M. S., Gierens, H., Nauck, M. A., Marz, W. and Wieland, H.: Rapid genotyping of human platelet antigen 1 (HPA-1) with fluorophore-labelled hybridization probes on the Lightcycler. Brit. J. Haematol. 105, pp. 803-810, 1999;

Pease, A. C., Solas, D., Sullivan, E. J., Cronin, M. T., Holmes, C. P. and Fodor, S. P. A.: Light-generated oligonucleotide arrays for rapid DNA sequence analysis. Proc. Natl. Acad. Sci. USA, 1994;

Southern, E. M.: DNA chips: Analysis sequence by hybridization to oligonucleotides on a large scale. Trends Genet. 12, pp. 110-115, 1996;

Syvanen, A. C., Aalto-Setala, K., Harju, L., Kontula, K. and Soderlund, H.: A primer-guided nucleotide incorporation assay in the genotyping of apolipoprotein E. Genomics 8, pp. 684-692, 1990;

Tyagi, S. and Kramer, F. R.: Molecular beacons: Probes that fluoresce upon hybridization. Nature Biotechnol. 14, pp. 303-308, 1996.

What is claimed is:

1. A method for separating nucleic acid molecules which have specific alleles, comprising:
(a) hybridizing a nucleic acid comprising a heterosequence site with at least one nucleic acid primer specific to the heterosequence site to form a hybridized nucleic acid sequence, wherein the at least one specific nucleic acid primer is capable of undergoing elongation only when hybridized to the heterosequence site;
(b) subjecting the hybridized nucleic acid sequence to conditions which permit elongation of the at least one nucleic acid primer; and
(c) separating the hybridized nucleic acid sequences which have undergone elongation from the nucleic acid sequences from unhybridized nucleic acid sequences and the nucleic acid primers which have not undergone elongation.

2. The method of claim 1, wherein the 3′ end of the at least one nucleic acid primer corresponds in position to a polymorphic base within the heterosequence site and the nucleic acid primer is capable of undergoing elongation only when the 3′ end of the at least one nucleic acid primer is complementary to and hybridized to the polymorphic base within the heterosequence site.

3. The method of claim 2, wherein one of the primer or one of the elongated primer is labeled with a detection molecule and (d) further comprises separating the hybridized nucleic acid sequences which have undergone elongation utilizing the detection molecule.

4. The method of claim 1, further comprising:
(d) amplifying the nucleic acid molecule comprising the heterosequence site prior to hybridization with the at least one nucleic acid primer; and
(e) identifying the heterosequence site.

5. The method of claim 1 wherein the heterosequence site comprises a single nucleotide polymorphism.

6. A kit for separating nucleic acid molecules which have specific alleles comprising instructions for carrying out the method of claim 1.

7. A method for separating a nucleic acid molecule which has a specific allele, comprising:
(a) hybridizing a nucleic acid comprising one or more heterosequence sites with at least one nucleic acid primer specific to the heterosequence site and a ligation primer to form a hybridized nucleic acid sequence, wherein the 3′ end of the at least one nucleic acid primer corresponds in position to a polymorphic base within the heterosequence site and the 5′ end of the ligation primer is adjacent to the 3′ end of the at least one nucleic acid primer;
(b) subjecting the at least one nucleic acid primer and the ligation primer to conditions which permit ligation of the at least one nucleic acid primer and the ligation primer; and
(c) separating the hybridized nucleic acid molecule in which the primers have undergone ligation.

8. The method of claim 7 wherein one of the at least one nucleic acid primer and the ligation primer is labeled with a detection molecule and (c) further comprises separating the hybridized nucleic acid sequences which have undergone elongation utilizing the detection molecule.

9. The method of claim 8 wherein the at least one nucleic acid primer comprises a plurality of primers having different sequences and each sequence is associated with a particular detection molecule such that no two sequences are associated with the same detection molecule.

10. A kit for separating a nucleic acid molecule which has a specific allele comprising instructions for carrying out the method of claim 7.

11. A method for separating a nucleic acid molecule which has a specific allele, comprising:
(a) hybridizing a nucleic acid comprising one or more heterosequence sites with at least one nucleic acid primer specific to the heterosequence site to form hybridized nucleic acid complexes; and
(b) separating the hybridized nucleic acid complexes which have complete complementary hybridization from the hybridized nucleic acid complexes which do not have complete complementary hybridization.

12. The method of claim 11 further comprising determining the sequence of the nucleic acid comprising the one or more heterosequence sites.

13. The method of claim 11 wherein step (b) comprises heating the hybridized nucleic acid complexes to a temperature at which the nucleic acid complexes which do not have complete complementary hybridization dissociate and which the nucleic acid complexes which have complete complementary hybridization do not dissociate.

14. The method of claim 11 wherein the at least one nucleic acid primer is labeled with a detection molecule.

15. A kit for separating a nucleic acid molecule which has a specific allele comprising instructions for carrying out the method of claim 11.

16. A method for separating a nucleic acid molecule which has a specific allele, comprising:
(a) hybridizing a nucleic acid comprising at least a 5′ heterosequence site and a 3′ heterosequence site with a hetero primer specific to the 3′ heterosequence site and a homo primer to form a hybridized nucleic acid sequence, wherein the 3′ end of the hetero primer corresponds in position to a polymorphic base within the 3′ heterosequence site, the homo primer is capable of hybridizing to the nucleic acid at a position located 5′ of the 5′ heterosequence site and the hetero primer is capable of undergoing elongation only when the 3′ end of the hetero primer is complementary to and hybridized to the polymorphic base within the 3′ heterosequence site;
(b) elongating the hybridized hetero primer such that the nucleic acid sequence between the hetero primer and the homo primer is produced and includes the 5′ heterosequence site; and
(c) determining the identity of the 5′ heterosequence site.

17. The method of claim 16 further comprising:
(d) utilizing the identity of the 5′ heterosequence site to produce another hetero primer and another homo primer, wherein the 3′ end of the another hetero primer corresponds in position to a polymorphic base within the 5′ heterosequence site, the 5′ heterosequence is located 3′ to another 5′ heterosequence, the homo primer is capable of hybridizing to the nucleic acid at a position located 5′ of the another 5′ heterosequence site and the hetero primer is capable of undergoing elongation only when the 3′ end of the hetero primer is complementary to and hybridized to the polymorphic base within the 5′ heterosequence site;
(e) hybridizing nucleic acid sequence with the another hetero primer and the another homo primer; and
(f) repeating steps (a) through (e) one or more times.

18. The method of 16 further comprising determining the haplotype of the nucleic acid molecule.

19. A kit for separating a nucleic acid molecule which has a specific allele comprising instructions for carrying out the method of claim 16.

20. A method for identifying an allele in a nucleic acid molecule, comprising:
(a) hybridizing a nucleic acid comprising a plurality of heterosequence sites with at least one primer to a produce a hybridized nucleic acid, wherein the at least one primer is attached to a bead;
(b) elongating the hybridized primer to produce an extended primer;
(c) dissociating the nucleic acid from the extended primer;
(d) hybridizing the extended primer with a second primer attached to the bead;
(e) elongating the second primer to produce a second extended primer; and
(f) identifying any heterosequence sites of the nucleic acid utilizing the extended primer, the second extended primer or both.

21. A method for identifying an allele in a nucleic acid molecule comprising instructions for carrying out the method of claim 20.
